(19) United States
(12) Patent Application Publication
(10) Pub. No.: US 2023/0121257 A1
(43) Pub. Date: Apr. 20, 2023

(54) COMPOSITION COMPRISING CARTILAGE INGREDIENT FOR REGENERATION OF CARTILAGE AND PREPARATION METHOD THEREFOR

(71) Applicant: L&C BIO Co., Ltd., Seongnam-si, Gyeonggi-do (KR)

(72) Inventors: Kee Won LEE, Mapo-gu Seoul (KR); Eun Seong LEE, Gwangmyeong-si, Gyeonggi-do (KR); Hyung Gu KIM, Nowon-gu Seoul (KR); Whan Chul LEE, Seongdong-gu Seoul (KR)

(21) Appl. No.: 17/795,930

(22) PCT Filed: Feb. 28, 2020
(86) PCT No.: PCT/KR2020/002894

§ 371 Date: Jul. 28, 2022

(51) Int. Cl. C08J 3/12; C08L 89/04; C08J 3/24

(52) U.S. Cl. CPC C08J 3/122; C08L 89/04; C08J 3/24; C08J 2389/04

(57) ABSTRACT

A composition includes a cartilage component for regeneration of cartilage and a manufacturing method therefor. A composition for regeneration of cartilage, in which a micronized cartilage powder is physically mixed with a biocompatible polymer or a chemically crosslinked biocompatible polymer. When applied, the composition can increase morphological retention and ease of use at cartilage injury sites.

### TECHNICAL FIELD

The present invention relates to a composition comprising a cartilage ingredient for regeneration of cartilage and a preparation method therefor.

### BACKGROUND ART

Osteoarthritis is a chronic disease characterized by pain, stiffness and loss of function due to damage to cartilage and surrounding tissues, and its incidence rate is increasing with an increasingly aging population. Cartilage is an aneural and avascular tissue, and when once damaged, surgical treatment is required because it is difficult to regenerate the cartilage and the damage progresses extensively.

As a currently applied surgical treatment, a method of improving cartilage injury lesions, such as microfracture, an osteochondral autograft transplantation system, or autologous chondrocyte implantation has been used. However, for microfracture, which is a primary procedure, cartilage may be easily washed with synovial fluid or lavage fluid to cause incomplete cartilage regeneration, and blood clots may easily be stripped away or worn out by body weight or joint motion. Further, microfracture is limitedly applied to small cartilage injury sites and exhibits a limited effect on complete cartilage regeneration due to the production of fibrocartilage rather than the hyaline cartilage, which is an original articular cartilage ingredient. Meanwhile, the osteochondral autograft transplantation system or autologous chondrocyte implantation shows a high success rate when applied to larger cartilage injury sites, but requires two procedures of autograft transplantation through a secondary procedure after collection of autogenous cartilage or autologous chondrocytes as a primary procedure, and there is always a problem of damage to normal tissue around the cartilage during the collection process.

A stem cell-based cartilage therapy that has recently attracted attention is a method of inducing cartilage production through the differentiation of stem cells into chondrocytes by injecting stem cells collected and isolated from autologous or allogeneic tissue into a cartilage injury site, and has no limitation in the applicable area of the lesion site. However, in the case of such autologous stem cells, it is essential to collect autologous cells for transplantation, and there is a limiting factor of high cost. In addition, injected stem cells are not completely guaranteed to differentiate into normal chondrocytes, requiring long-term hospitalization and rehabilitation after the procedure.

In order to solve the limitations of the existing treatment methods mentioned above, a method of directly applying a human-derived cartilage ingredient to a cartilage injury site has been applied. Such a procedure may improve the regeneration of cartilage by processing cartilage collected from a post-mortem cartilage tissue donor into particles and granules or micronized powder forms, and then mixing the processed cartilage with sterile physiological saline or a patient's blood or platelet-rich plasma (PRP) and injecting the resulting mixture into a cartilage injury site.

Among commercially available products used in such a procedure, DeNovo-NT® (Zimmer Biomet) is a human body tissue product in which cartilage tissue obtained from the femoral condyle of a post-mortem donor from an infant to the age of under 13 is processed into particles with an average particle size of 500 to 1000 μm and packaged in a state of being immersed in a sterile saline solution. Previous clinical studies reported that as a result of applying DeNovo-NT® to a group of patients with Outerbridge grade IV articular cartilage loss in the patella, normal or similar cartilage production was exhibited.

Another commercially available product, BioCartilage® (Arthrex, Inc.), is a human tissue product made by processing donor cartilage tissue into micronized powder with an average particle size of 100 to 300 μm. Preclinical animal model study results reported that when the product was mixed with PRP after microfracture and used for a chondral defect, not only safety and biocompatibility, but also improved cartilage regeneration compared to microfracture alone were exhibited.

As in the above two cases, a method of using human-derived cartilage directly on the cartilage injury site of a patient has the following advantages over existing treatment methods:

\textcircled{1} By supplementing a cartilage injury site with a human-derived hyaline cartilage ingredient, which is an inherent articular cartilage ingredient, rapid cartilage regeneration may be induced through homogeneous components.

\textcircled{2} When microfracture is performed alone, the limitation of fibrous cartilage production at the treatment site may be overcome.

\textcircled{3} There is no procedure step for collecting autologous cartilage tissue, autologous cells, and autogenous periosteum from a patient.

\textcircled{4} It is cheaper than the existing cartilage treatment method using stem cells.

Meanwhile, among the human-derived cartilage therapeutic materials in the above two cases, DeNovo-NT® has a limitation in the acquisition of therapeutic material, in which human cartilage needs to be obtained from a post-mortem donor from an infant or the age under 13. Furthermore, since the therapeutic material itself includes chondrocytes of living humans, there is an inherent possibility of causing an immune response after the procedure. The shelf life of therapeutic materials is very short, within a few weeks, and the distribution method is also very limited. Further, since cartilage particles are simply provided in the form of being hydrated in sterile physiological saline, it is difficult to maintain the morphology at a cartilage injury site during surgery, so there is a disadvantage in that user accessibility is also very limited.

Since a final formulation of BioCartilage®, which is the other human-derived cartilage treatment material of the above two cases, is also provided as a dehydrated cartilage powder, the product cannot be applied directly to a cartilage injury site, so the product is associated with the inconvenience of having to be used after a medical specialist manually mixes the product with the patient's blood or PRP during the procedure to secure viscosity.

### RELATED ART DOCUMENT

### Patent Documents

1. US Patent Publication No. 2012-0239146
2. US Patent Publication No. 2013-0338792

### Non-Patent Documents

1. Tompkins M. et al., Preliminary results of a novel single-stage cartilage restoration technique: Particulated juvenile articular cartilage allograft for chondral defects of the patella. Arthroscopy, 2013, 29(10), pp. 1661-1670.
2. Fortier L. A. et al., BioCartilage improves cartilage repair compared with microfracture alone in an equine model of full-thickness cartilage loss. Am J Sports Med, 2016, 44(9), pp. 2366-2374.

### DISCLOSURE

### Technical Problem

Thus, an object of the present invention is to provide a method for preparing micronized cartilage powder by crushing and sieve-separating cartilage.

In addition, another object of the present invention is to provide a composition for regeneration of cartilage capable of maximizing the cartilage regeneration effect because when a micronized cartilage powder according to the present invention is transplanted into the body, the micronized cartilage powder is transplanted in a state of being well aggregated in a cartilage injury site without being scattered in a micronized powder, particle or granular state.

### Technical Solution

The present invention provides a method for preparing a micronized cartilage powder, the method comprising: a lyophilization step of lyophilizing cartilage;

a crushing step of crushing the lyophilized cartilage; and

a sieve-separation step of sieve-separating the crushed cartilage.

Furthermore, the present invention provides a composition for regeneration of cartilage, comprising: a micronized cartilage powder prepared by the above-described preparation method; and

a biocompatible polymer or a crosslinked product of the biocompatible polymer.

Further, the present invention provides a method for preparing a composition for regeneration of cartilage, the method comprising: mixing a micronized cartilage powder prepared by the above-described preparation method; and a biocompatible polymer or a crosslinked product of the biocompatible powder.

### Advantageous Effects

The present invention can provide a composition for treating cartilage, which is capable of minimizing the remaining immune response-inducing factors and inducing homogenous cartilage tissue to be safely and effectively regenerated, by using a micronized cartilage powder prepared by crushing and sieve-separating allogeneic or xenogeneic cartilage, followed by delipidation and decellularization.

In addition, the present invention provides a composition for regeneration of cartilage, in which a micronized cartilage powder is physically mixed with a biocompatible polymer or a chemically crosslinked biocompatible polymer, whereby when applied, the composition can increase morphological retention and ease of use at cartilage injury sites.

Specifically, since the composition for regeneration of cartilage according to the present invention is prepared by mixing a micronized cartilage powder with a hydrogel-like excipient, the composition may be transplanted into a cartilage injury site in a well-aggregated state without being scattered in a powder, particle or granule state to maximize the cartilage regeneration effect.

### DESCRIPTION OF DRAWINGS

FIG. 1 is (A) a set of photographs illustrating the process of obtaining a micronized cartilage powder through crushing and sieve-separation and (B) a graph measuring the particle size distribution of the prepared micronized cartilage powder.

FIG. 2 is a graph quantifying the contents of collagen and sulfated glycosaminoglycan (sGAG) measured in the micronized cartilage powder.

FIG. 3 is the result of measuring the complex viscosity of a composition according to a mixing ratio of the micronized cartilage powder and an HA-CMC excipient and a set of photographs illustrating a shape after the syringe output of a composition for regeneration of cartilage prepared at a mixing ratio having the maximum complex viscosity, and a morphological retention ability through handling.

FIG. 4 is a set of photographs illustrating a composition for regeneration of cartilage at knee articular cartilage injury sites of rabbits before and after the composition is transplanted.

FIG. 5 is a set of photographs illustrating knee articular cartilage injury sites of rabbits 12 weeks after transplantation of a composition for regeneration of cartilage and the composition after the knee articular cartilage injury sites are extracted, and a set of photographs illustrating staining with H&E, Safranin-O/Fastgreen and Masson's trichrome for histological analysis.

### MODES OF THE INVENTION

The present invention provides a method for preparing a micronized cartilage powder, the method comprising: a lyophilization step of lyophilizing cartilage;

a crushing step of crushing the lyophilized cartilage; and

a sieve-separation step of sieve-separating the crushed cartilage.

In exemplary embodiments of the present invention, a micronized cartilage powder was prepared and a composition for regeneration of cartilage, comprising the micronized cartilage powder and a crosslinked product (an HA-CMC excipient) of a biocompatible polymer was also prepared, and thus it was confirmed that the composition for regeneration of cartilage had excellent viscoelastic characteristics. Further, it was confirmed that, by performing an in vivo experiment on the composition for regeneration of cartilage, the composition had an excellent cartilage regeneration effect compared to the case where only microfracture in the related art was performed.

Hereinafter, the method for preparing a micronized cartilage powder according to the present invention will be described in more detail.

As used herein, the micronized cartilage powder (hereinafter, it may be referred to as cartilage powder.) refers to cartilage having a size of several μm prepared by the preparation process according to the present invention. The cartilage powder may be used in a sense that it includes not only powder in a dictionary sense but also particles and granules.

The method for preparing a micronized cartilage powder of the present invention comprises a lyophilization step; a crushing step; and a sieve-separation step.

In an exemplary embodiment, the cartilage may be allogeneic or xenogeneic cartilage. The allogeneic cartilage means human-derived cartilage, and the xenogeneic cartilage may mean cartilage derived from animals other than humans, that is, mammals such as pigs, cows, and horses. In the present invention, human-derived cartilage donated after death may be used.

In the present invention, a washing step may be performed before performing the lyophilization step, and sterile distilled water may be used as a washing solvent. Impurities in cartilage may be removed through the step.

In addition, in the present invention, it is possible to carry out a step of removing a soft tissue and the perichondrium from the cartilage before performing the lyophilization step.

In an exemplary embodiment, cartilage tissue and the perichondrium may be removed using a blade and a rongeur. Specifically, after a cartilage-to-cartilage interface is vertically cut with a blade, the perichondrium may be removed by pulling the corners of the cut surface in a state in which the tissue surface is moistened with sterile distilled water to prevent the tissue surface from drying out using a rongeur.

In the present invention, the lyophilization step is a step of lyophilizing cartilage.

In an exemplary embodiment, the cartilage may be cartilage on which the above-described washing is performed and from which the cartilage tissue and perichondrium are removed.

In an exemplary embodiment, lyophilization is a method of absorbing moisture in a vacuum after rapidly cooling tissue (cartilage) in a frozen state, and moisture in the cartilage may be adjusted by performing the aforementioned lyophilization.

In an exemplary embodiment, lyophilization may be performed at −50 to −80° C. for 24 to 96 hours.

In the present invention, the crushing step is a step of crushing the lyophilized cartilage.

In an exemplary embodiment, crushing may be performed using a tissue grinder. In this case, a crushing time may be 30 seconds to 5 hours. In an exemplary embodiment, the cartilage after crushing may have a particle diameter of 1 to 1000 μm.

The crushing may be performed one or more times.

In the present invention, the sieve-separation step is a step of sieve-separating the crushed cartilage in the crushing step.

In an exemplary embodiment, sieve separation may be performed using a sieve with a mesh of 100 to 1,000 μm.

The sieve separation may be performed one or more times.

In the present invention, after the sieve-separation step is performed, a delipidation step of subjecting the micronized cartilage powder to delipidation; and a decellularization step of subjecting the micronized cartilage powder to decellularization may be additionally performed.

In the present invention, the delipidation step is a step of removing lipid components from adipose tissue.

In an exemplary embodiment, delipidation refers to the removal of lipid components from tissue.

The lipid components may be removed by chemical treatment.

In an exemplary embodiment, the type of chemical treatment is not particularly limited, and may be performed using a delipidation solution. The delipidation solution may include a polar solvent, a non-polar solvent or a mixed solvent thereof. Water, alcohol or a mixed solution thereof may be used as the polar solvent, and methanol, ethanol or isopropyl alcohol may be used as the alcohol. Hexane, heptane, octane, or a mixed solution thereof may be used as the non-polar solvent. Specifically, in the present invention, a mixed solution of isopropyl alcohol and hexane may be used as the delipidation solution. In this case, a mixing ratio of isopropyl alcohol and hexane may be 40:60 to 60:40.

The treatment time of the delipidation solution may be 1 to 30 hours, 1 to 20 hours or 10 to 20 hours.

In the present invention, the delipidation step is a step of removing cells from a cartilage powder from which lipid components are removed.

In an exemplary embodiment, decellularization refers to the removal of other cellular components, for example, nuclei, cell membranes, nucleic acids, and the like other than the extracellular matrix from tissue.

In an exemplary embodiment, decellularization may be performed using a basic solution, and as the basic solution, it is possible to use one or more selected from the group consisting of sodium hydroxide, potassium hydroxide, ammonium hydroxide, calcium carbonate, magnesium hydroxide, calcium hydroxide and ammonia. In the present invention, sodium hydroxide (NaOH) may be used as the basic solution.

In an exemplary embodiment, a concentration of the basic solution may be 0.01 to 1 N, 0.06 to 0.45 N, 0.06 to 0.2 N, or 0.08 to 1.02 N. Within the above concentration range, cells are easily removed.

Further, in an exemplary embodiment, the decellularization step may be performed for 40 to 60 minutes, 70 to 200 minutes, or 90 to 150 minutes. Within the above time range, cells are easily removed.

In the present invention, after the decellularization step is performed, if necessary, it is possible to further perform one or more steps selected from the group consisting of a neutralization step of neutralizing the cartilage powder with an acidic solution;

a washing step of washing the neutralized cartilage powder;

a step of centrifuging the washed cartilage powder; and

a step of lyophilizing the centrifuged cartilage powder.

In an exemplary embodiment, through the centrifuging step, impurities in the delipidation step and the decellularization step may be removed, and a micronized cartilage powder (precipitate) with high purity may be obtained.

In addition, during the washing step, sterile distilled water and/or 70% ethanol may be used as a washing solution.

In an exemplary embodiment, centrifugation may be performed at 4,000 to 10,000 rpm, or 8,000 rpm for 5 to 30 minutes, 5 to 20 minutes, or 10 minutes.

In an exemplary embodiment, lyophilization may be performed at −50 to −80° C. for 24 to 96 hours.

Furthermore, the present invention relates to a micronized cartilage powder prepared by the above-described method for preparing a micronized cartilage powder.

In an exemplary embodiment, the micronized cartilage powder may have an average particle diameter of 100 μm to 900 μm, 300 to 800 μm or 400 to 700 μm, and an average particle size of 100 μm to 900 μm.

The micronized cartilage powder of the present invention may include collagen and glycosaminoglycan (GAG), which are the main constituents of the articular cartilage extracellular matrix. The content of collagen may be 30 wt % or more, 40 to 90 wt %, 50 to 80 wt % or 60 to 80 wt %, and the content of glycosaminoglycan may be 5 wt % or more, 5 to 40 wt % or 10 to 30 wt %, concerning the total weight (100 wt %) of the micronized cartilage powder.

Further, the present invention relates to a composition for regeneration of cartilage, comprising a micronized cartilage powder prepared by the above-described preparation method.

The composition for regeneration of cartilage according to the present invention may comprise a biocompatible polymer or a crosslinked product of the biocompatible polymer along with the micronized cartilage powder.

In an exemplary embodiment, as the micronized cartilage powder, a cartilage powder prepared by the above-described preparation method may be used, and an average particle diameter thereof may be 100 μm to 900 μm. Within the above particle diameter range, the micronized cartilage powder is suitable for bioinjection and can be injected with a syringe. In addition, the micronized cartilage powder includes collagen and glycosaminoglycan (GAG), which are the main constituents of the articular cartilage extracellular matrix, and thus, has a very good cartilage regeneration effect.

In an exemplary embodiment, the content of the micronized cartilage powder may be 10 to 90 parts by weight, 10 to 30 parts by weight or 20 to 30 parts by weight concerning the total weight (100 parts by weight) of the composition. Within the above range, the micronized cartilage powder can be injected with a syringe, and may have an excellent cartilage regeneration ability.

In the present invention, the biocompatible polymer or the crosslinked product of the biocompatible polymer may improve the viscoelastic characteristics of the composition for regeneration of cartilage, and may improve volume retention ability in the body. In the present invention, such a biocompatible polymer or a crosslinked product of the biocompatible polymer may be referred to as an excipient.

In an exemplary embodiment, the biocompatible polymer or the crosslinked product of the biocompatible polymer may be present in the composition in a hydrogel state, which may be referred to as a hydrogel excipient.

In an exemplary embodiment, the crosslinked product of the biocompatible polymer refers to one or more chemically crosslinked biocompatible polymers.

In an exemplary embodiment, the biocompatible polymer or the crosslinked product of the biocompatible polymer may have a molecular weight of 10 kDa to 2 MDa.

In an exemplary embodiment, as the biocompatible polymer, it is possible to use one or more selected from the group consisting of collagen, sodium hyaluronate, chitosan, sodium carboxymethyl cellulose, alginate and gelatin.

In an exemplary embodiment, the crosslinked product of the biocompatible polymer may be a crosslinked product of one or more biocompatible polymers selected from the group consisting of collagen, sodium hyaluronate, chitosan, sodium carboxymethyl cellulose, alginate and gelatin. Specifically, in the present invention, a crosslinked product of sodium hyaluronate (HA) and sodium carboxymethyl cellulose (CMC) may be used.

In an exemplary embodiment, the biocompatible polymer is crosslinked by a crosslinking agent, and the crosslinking agent may be one or more selected from the group consisting of 1,4-butanediol diglycidyl ether (BDDE), ethylene glycol diglycidyl ether (EGDGE), 1,6-hexanediol diglycidyl ether, propylene glycol diglycidyl ether, polypropylene glycol diglycidyl ether, polytetramethylene glycol diglycidyl ether, neopentyl glycol diglycidyl ether, polyglycerol polyglycidyl ether, diglycerol polyglycidyl ether, glycerol polyglycidyl ether, tri-methylpropane polyglycidyl ether, 1,2-bis(2,3-epoxypropoxy)ethylene, pentaerythritol polyglycidyl ether, and sorbitol polyglycidyl ether.

In an exemplary embodiment, the content of the biocompatible polymer or the crosslinked product of the biocompatible polymer may be 10 to 90 parts by weight, 20 to 80 parts by weight, or 50 to 80 parts by weight concerning the total weight (100 parts by weight) of the composition. Within the above range, physical properties of the biocompatible polymer may be improved, and volume retention ability in the body may also be improved.

In an exemplary embodiment, the composition for regeneration of cartilage may have a complex viscosity of 5,000 to 100,000 Pa·s. The complex viscosity refers to a result value measured by a rotational rheometer (frequency: 0.1 to 100 Hz, temperature: 25° C., and strain: 1%).

The complex viscosity is a frequency-dependent viscosity calculated by a vibration measurement method, and the above values are values in which G″ (viscous modulus (loss modulus)), G′ (elastic modulus (storage modulus)) and the value of frequency to be measured are reflected. In the present invention, the composition for regeneration of cartilage may have a complex viscosity of 20,000 to 5,000 Pa·s or 35,000 to 45,000 Pa·s.

In an exemplary embodiment, the composition for regeneration of cartilage of the present invention may be injected or inserted in vivo through injection with a syringe, and the like. Such a composition for regeneration of cartilage may be used as a general medical material.

Furthermore, the present invention relates to a method for preparing the above-described composition for regeneration of cartilage.

The method for preparing the composition for regeneration of cartilage comprises: a step of mixing a micronized cartilage powder; and a biocompatible polymer or a crosslinked product of the biocompatible polymer.

In the present invention, as the micronized cartilage powder, a cartilage powder prepared by the above-described preparation method may be used.

Specifically, the micronized cartilage powder may be prepared by a lyophilization step of lyophilizing cartilage;

a crushing step of crushing the lyophilized cartilage;

a sieve-separation step of sieve-separating the crushed cartilage;

a delipidation step of subjecting the micronized cartilage powder to delipidation; and

a decellularization step of subjecting the micronized cartilage powder to decellularization.

In the present invention, as the biocompatible polymer or the crosslinked product of the biocompatible polymer, a commercially available product on the market may be used. Further, the crosslinked product may be prepared in a laboratory and the like using a biocompatible polymer and used.

The crosslinked product of the biocompatible polymer may be prepared by a crosslinking step of crosslinking a biocompatible polymer using a crosslinking agent; and

a lyophilization step of lyophilizing the crosslinked product.

In the present invention, the crosslinking step is a step of crosslinking a biocompatible polymer using a crosslinking agent. In the above step, the above-described types may be used as the biocompatible polymer and the crosslinking agent.

In an exemplary embodiment, the biocompatible polymers may be bonded through an amide bond.

In an exemplary embodiment, the content of the crosslinking agent may be 0.5 to 10 parts by weight with concerning the biocompatible polymer.

In the present invention, a step of washing the crosslinked reaction product may be further performed before performing lyophilization. In this case, phosphate-buffered saline (PBS) and/or sterile distilled water may be used as a washing solution.

In the present invention, the lyophilization step is a step of lyophilizing the crosslinked biocompatible polymer in the above step.

In an exemplary embodiment, lyophilization may be performed at −50 to −80° C. for 24 to 96 hours.

In the present invention, before the lyophilized crosslinked product is mixed with the cartilage powder, a hydrogel may be formed by mixing the crosslinked product with a solvent such as sterile physiological saline.

In the present invention, a micronized cartilage powder, and a biocompatible polymer or a crosslinked product of the biocompatible polymer may be mixed by physical mixing.

In this case, the biocompatible polymer or the crosslinked product of the biocompatible polymer may be in the form of a hydrogel.

In an exemplary embodiment, the content of the micronized cartilage powder in the physically mixed mixture may be 10 to 90 parts by weight, 10 to 30 parts by weight or 20 to 30 parts by weight.

In addition, the content of the biocompatible polymer or the crosslinked product of the biocompatible polymer in the mixture may be 10 to 90 parts by weight, 20 to 80 parts by weight, or 50 to 80 parts by weight.

In an exemplary embodiment, the mixture may be prepared by dissolving the crosslinked product of the lyophilized biocompatible polymer in a solvent, and then mixing the resulting solution with the micronized cartilage powder. In this case, physiological saline may be used as the solvent.

The present invention may further comprise a step of sterilizing the mixture. Through the sterilization step, the immunity in the composition for regeneration of cartilage may be removed, and bacteria and the like may be effectively destroyed.

In an exemplary embodiment, the sterilization step may be performed by irradiating the composition with radiation, and the irradiation range of radiation may be 10 to 30 kGy.

The composition for regeneration of cartilage prepared in the present invention may have a paste form.

Furthermore, the present invention relates to a use of the above-described composition for regeneration of cartilage.

The composition for regeneration of cartilage according to the present invention may induce the regeneration of cartilage after transplantation in the body, also has improved viscoelastic characteristics, and thus has an effect of having excellent volume retention ability in the body.

Therefore, in an exemplary embodiment, the composition for regeneration of cartilage of the present invention may be injected or inserted in vivo through injection with a syringe, and the like.

The present invention will be described in more detail through the following Examples. However, it is to be understood by a person with ordinary skill in the art that the scope of the present invention is not limited to the following examples, and various alterations, modifications or applications can be made within a scope not deviating from the technical details derived by the details described in the accompanying claims.

### EXAMPLES

### Example 1: Preparation of Micronized Cartilage Powder

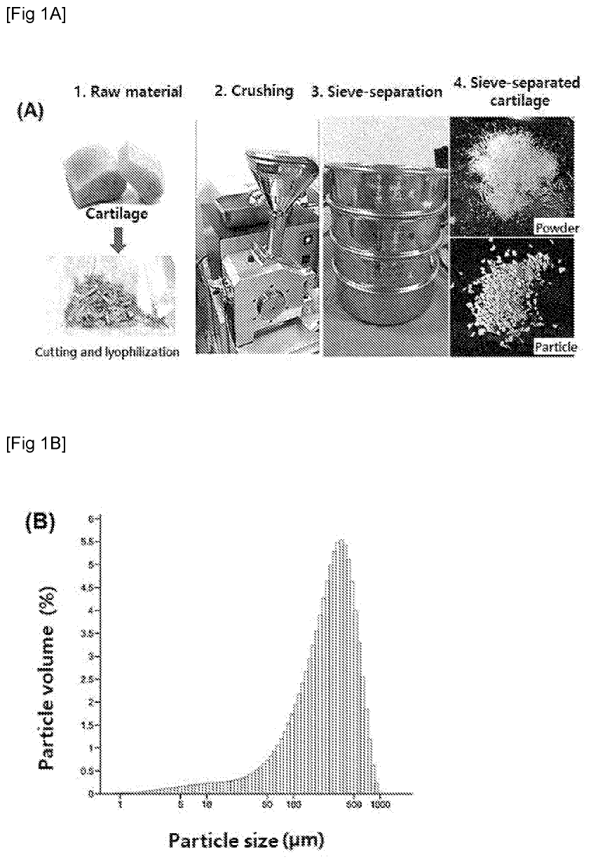

Human-derived cartilage was washed with sterile distilled water, soft tissue and the perichondrium were removed and cut, and then the cartilage was pre-treated by a lyophilization process. The lyophilized cartilage was ground by a tissue grinder (power cutting mill, Pulverisette25, FRITSCH, Germany) for 30 seconds to 5 hours and sieve-separated through a sieve with a mesh of 100 to 1000 μm. A human-derived micronized cartilage powder was obtained by performing the grinding and sieve-separating process several times (see FIG. 1A).

### Experimental Example 1. Analysis of Average Particle Size of Micronized Cartilage Powder

(1) Method

The particle size of the micronized cartilage powder prepared in Example 1 was analyzed.

After 40 mL of sterile distilled water was added to 1 g of the micronized cartilage powder and the resulting mixture was shaken and dispersed for 2 minutes, an average particle size was measured in a range of 0.017 to 2,000 μm in a wet mode using a particle size analyzer (LS 13 320, Beckman Coulter).

(2) Result

The results are illustrated in FIG. 1B.

As illustrated in FIG. 1B, it can be confirmed that the size of the micronized cartilage powder is distributed from 1 μm or more to 1000 μm, and an average particle size is in a range of 100 to 900 μm.

### Experimental Example 2. Component Analysis of Micronized Cartilage Powder

(1) Method

In order to confirm the main components and contents of the articular cartilage extracellular matrix contained in the micronized cartilage powder prepared in Example 1, the contents of collagen and GAG were measured.

First, to measure the content of collagen, the micronized cartilage powder was treated with a protease (proteinase K), and reacted with a 12.1 N hydrochloric acid (HCl, 35.0 to 37.0%, JUNSEI Chemical) solution at 110° C. for 16 hours or more, and then the content of collagen was quantified by a hydroxyproline analysis method.

Further, to measure the content of GAG, after the micronized cartilage powder was reacted with proteinase K at 60° C. for 16 hours, the content of GAG was quantified using a 1,9-dimethylmethylene blue (DMMB) analysis method.

(2) Result

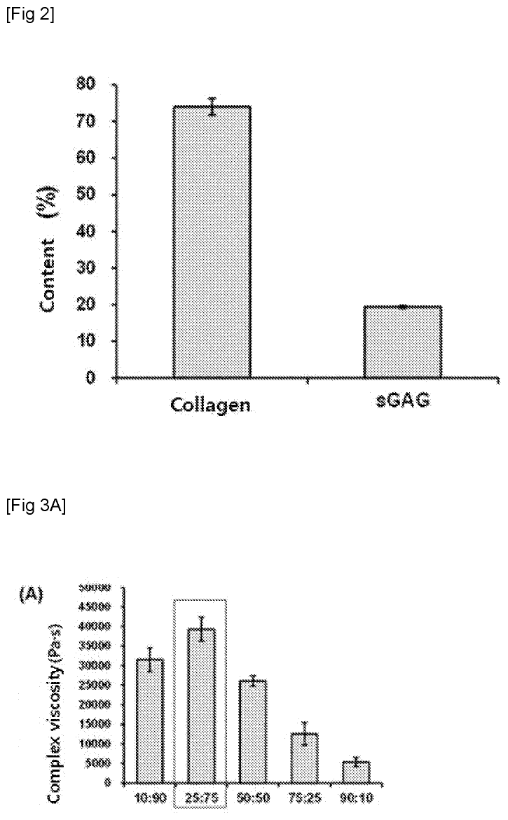

The results are illustrated in FIG. 2.

As illustrated in FIG. 2, it can be confirmed that the contents of collagen and sGAG in the micronized cartilage powder are 73.95 wt % and 19.39 wt %, respectively, concerning the total weight.

### Example 2. Preparation of Composition for Regeneration of Cartilage, Comprising Micronized Cartilage Powder and Chemically Crosslinked Biocompatible Polymer

### (1) Delipidation and Decellularization of Micronized Cartilage Powder

First, the micronized cartilage powder prepared in Example 1 was delipidated and decellularized.

The micronized cartilage powder was subjected to a delipidation process for 1 to 20 hours using 40% to 60% isopropyl alcohol and 40% to 60% hexane. Cells were removed by treating a tissue from which fat had been removed with 0.1 N sodium hydroxide (NaOH).

### (2) Preparation of Chemically Crosslinked Biocompatible Polymer

An HA-CMC excipient was prepared by mixing sodium hyaluronate (HA), which is a bio-derived polymer composed of N-acetylglucosamine and glucuronic acid, and sodium carboxymethyl cellulose (CMC), which is a vegetable polymer, with 1,4-butanediol diglycidyl ether (BDDE, Sigma-Aldrich), which is a crosslinking agent.

A reaction solvent was prepared by adding 1 mL to 5 mL (1 vol % to 5 vol %) of BDDE per 100 mL of a NaOH solution having a concentration of 0.1 N to 1 N. After 1 wt % to 10 wt % of HA and CMC were each added to the prepared reaction solvent, a mixed solution was prepared by homogeneously mixing the resulting mixture. The mixed solution was crosslinked by heating the mixed solution at 50° C. for 3 hours. The reaction product for which the crosslinking reaction had been completed was put into a dialysis membrane and dialyzed with 5 L of PBS at room temperature. After 2 hours, 5 L of PBS was replaced with 5 L of 50% EtOH, and the reaction product was dialyzed at room temperature for 1 hour. Thereafter, the reaction product was dialyzed with sterile distilled water at room temperature for 72 hours and lyophilized to finally obtain an HA-CMC excipient.

### (3) Preparation of Composition for Regeneration of Cartilage in Paste Form

A composition for regeneration of cartilage in paste form was mixed so as to contain a micronized cartilage ingredient in an amount of 10 wt % to 90 wt % concerning the total weight of the composition.

The crosslinked HA-CMC excipient that completed lyophilization was mixed with sterile physiological saline and gelled. A composition for regeneration of cartilage in paste form was prepared by finally mixing a micronized cartilage ingredient with the gelled HA-CMC excipient.

After a prefilled syringe was filled with the composition for regeneration of cartilage, the composition was sterilized with gamma rays.

### Experimental Example 3. Measurement of Complex Viscosity According to Mixing Ratio of Micronized Cartilage Powder and HA-CMC Excipient

(1) Method

The viscosities of compositions for regeneration of cartilage prepared according to the mixing ratio of the micronized cartilage powder prepared in (1) of Example 2 and the gelled HA-CMC excipient prepared in (2) of Example 2 were compared.

Specifically, a complex viscosity at 1 Hz was measured under conditions of frequency: 0.1 to 100 Hz, strain: 1% and temperature: 25° C. using a rotational rheometer (DHR-1, TA Instruments).

(2) Result

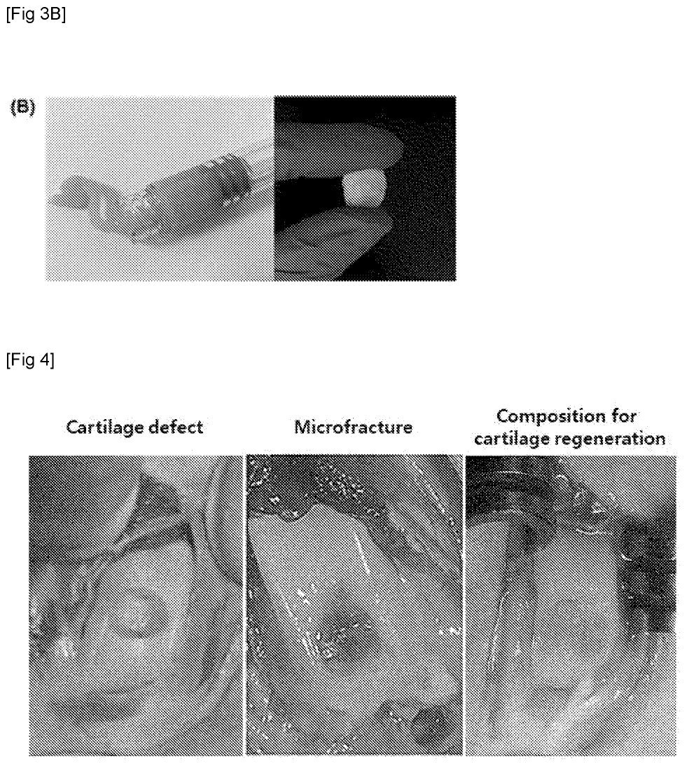

Measurement results are shown in FIG. 3 and the following Table 1.

TABLE 1

| | Micronized cartilage:Hydrogel of HA-CMC | | | | |
|---|---|---|---|---|---|
| | crosslinked product (wt %:wt %) | | | | |
| | 10:90 | 25:75 | 50:50 | 75:25 | 90:10 |
| Complex viscosity | 31555 ± | 39308 ± | 26089 ± | 12572 ± | 5381 ± |
| (Pa · s) (mean ± SD) | 3046 | 3050 | 1344 | 2854 | 1106 |

As shown in FIG. 3 and Table 1, it can be confirmed that the complex viscosity of the composition for regeneration of cartilage increases at a mixing ratio of 10:90 to 25:75, but thereafter, the complex viscosity decreases as the micronized cartilage powder increases and the HA-CMC excipient decreases.

Therefore, 25:75, which exhibits the highest complex viscosity, was selected as the final mixing ratio for the composition for regeneration of cartilage.

FIG. 3B illustrates the results of outputting the composition for regeneration of cartilage produced at the selected mixing ratio (25:75) from the syringe, and it can be confirmed that the composition maintains the paste form and is well aggregated during handling to maintain a morphology.

### Experimental Example 4. Verification of In Vivo Performance

(1) Method

The in vivo performance of the composition for regeneration of cartilage (mixing ratio 25:75) prepared in Example 2 was verified.

In this case, as a control, a cartilage defect group in which nothing was transplanted into the cartilage injury site and a group in which only microfracture was performed (microfracture group) were used.

The verification was performed as an experiment using New Zealand white rabbits (male, 18 weeks old, 2.5 kg).

After a full-thickness osteochondral defect with a diameter of 5 mm and a depth of 2 mm was generated in the trochlear grooves of the left and right knee articular joints of the experimental animals, microfracture was performed using a K-wire. The composition paste for regeneration of cartilage was transplanted into a site in which microfracture was performed and fibrin glue was applied. In order to confirm a cartilage repair and regeneration effect, results were analyzed by staining tissue after sacrificing the experimental animals 12 weeks after transplantation.

(2-1) Verification of Morphological Retention Ability at Cartilage Injury Site

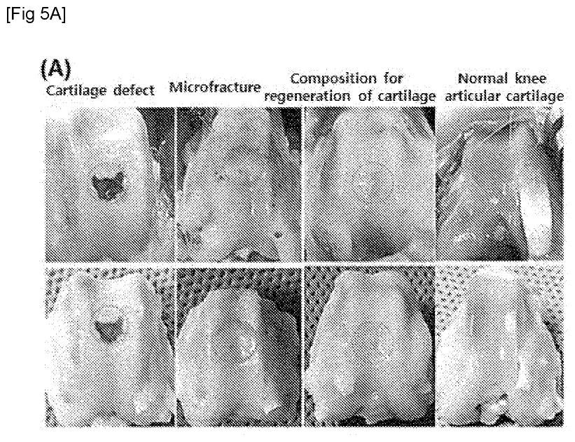

FIG. 4 is a set of photographs illustrating a composition for regeneration of cartilage at knee articular cartilage injury sites of rabbits before and after the composition is transplanted. In FIG. 4, immediately after transplanting the composition for regeneration of cartilage, the morphological retention ability of the composition for regeneration of cartilage can be confirmed by visually examining the cartilage injury site.

As illustrated in FIG. 4, it can be confirmed through visual examination that the cartilage injury site is filled with the composition for regeneration of cartilage having the paste form and maintained.

(2-2) Verification of Cartilage Repair and Regeneration Effect

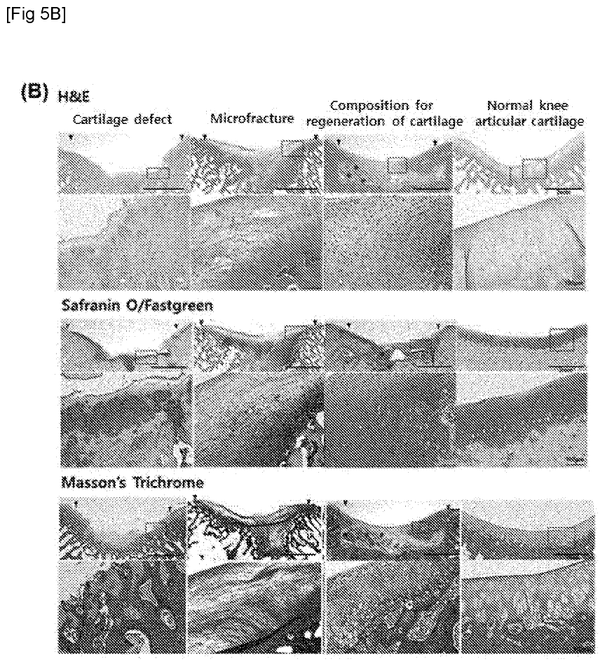

FIG. 5A is a set of photographs illustrating knee articular cartilage injury sites of rabbits 12 weeks after transplantation of a composition for regeneration of cartilage and the composition after the knee articular cartilage injury sites are extracted. In FIG. 5A, appearance continuity with adjacent normal cartilage can be observed by visually examining a transplanted site.

As illustrated in FIG. 5A, as a result of visual examination, it can be confirmed that the injury site into which the composition for regeneration of cartilage is transplanted has better continuity in appearance with adjacent normal cartilage than the control.

Meanwhile, FIG. 5B is a set of photographs illustrating staining with H&E, Safranin-O/Fastgreen and Masson's trichrome for histological analysis. In FIG. 5B, a cartilage repair effect can be confirmed by performing H&E, Safranin-O/Fastgreen and Masson's trichrome staining on the extracted sample.

As illustrated in FIG. 5B, it can be confirmed that the composition for regeneration of cartilage exhibits cell, GAG, and collagen production similar to normal knee articular cartilage, compared to the control. Through this, it can be confirmed that the composition for regeneration of cartilage according to the present invention has a relatively good cartilage regeneration effect.

1. A method for preparing a micronized cartilage powder, the method comprising:
a lyophilization step of lyophilizing cartilage;
a crushing step of crushing the lyophilized cartilage; and
a sieve-separation step of sieve-separating the crushed cartilage.

2. The method of claim 1, wherein crushing is performed for 30 seconds to 5 hours using a tissue grinder.

3. The method of claim 1, wherein sieve-separation is performed using a sieve with a mesh size of 100 to 1,000 μm.

4. The method of claim 1, further comprising: after the sieve-separation step is performed, a delipidation step of subjecting the micronized cartilage powder to delipidation; and
a decellularization step of subjecting the micronized cartilage powder to decellularization.

5. A composition for regeneration of cartilage, comprising: a micronized cartilage powder prepared by the preparation method according to claim 1; and
a biocompatible polymer or a crosslinked product of the biocompatible polymer.

6. The composition of claim 5, wherein the micronized cartilage powder has an average particle diameter of 100 to 900 μm.

7. The composition of claim 5, wherein the micronized cartilage powder comprises 30 wt % or more of collagen and 5 wt % or more of glycosaminoglycan.

8. The composition of claim 5, wherein a content of the micronized cartilage powder is 10 to 90 parts by weight concerning the total weight of the composition.

9. The composition of claim 5, wherein the biocompatible polymer comprises one or more selected from the group consisting of collagen, sodium hyaluronate, chitosan, sodium carboxymethyl cellulose, alginate and gelatin, and
the crosslinked product of the biocompatible polymer is a crosslinked product of one or more biocompatible polymers selected from the group consisting of collagen, sodium hyaluronate, chitosan, sodium carboxymethyl cellulose, alginate and gelatin.

10. The composition of claim 9, wherein the biocompatible polymer is crosslinked by a crosslinking agent, and
the crosslinking agent is one or more selected from the group consisting of 1,4-butanediol diglycidyl ether (BDDE), ethylene glycol diglycidyl ether (EGDGE), 1,6-hexanediol diglycidyl ether, propylene glycol diglycidyl ether, polypropylene glycol diglycidyl ether, polytetramethylene glycol diglycidyl ether, neopentyl glycol diglycidyl ether, polyglycerol polyglycidyl ether, diglycerol polyglycidyl ether, glycerol polyglycidyl ether, tri-methylpropane polyglycidyl ether, 1,2-bis(2,3-epoxypropoxy)ethylene, pentaerythritol polyglycidyl ether, and sorbitol polyglycidyl ether.

11. The composition of claim 5, wherein a content of the biocompatible polymer or the crosslinked product of the biocompatible polymer is 10 to 90 parts by weight concerning the total weight of the composition.

12. The composition of claim 5, wherein the composition has a complex viscosity of 5,000 to 100,000 Pa·s.

13. A method for manufacturing a composition for regeneration of cartilage, the method comprising: mixing the micronized cartilage powder prepared by the preparation method according to claim 1; and a biocompatible polymer or a crosslinked product of the biocompatible polymer.

14. The method of claim 13, wherein the crosslinked product of the biocompatible polymer is prepared by a crosslinking step of crosslinking a biocompatible polymer using a crosslinking agent; and
a lyophilization step of lyophilizing the crosslinked product.
